(19) United States
(12) Patent Application Publication
(10) Pub. No.: US 2024/0226005 A1
(43) Pub. Date: Jul. 11, 2024

(54) NANO- AND MICRO-FORMULATIONS

(71) Applicant: Huxley Health Inc., Concord (CA)

(72) Inventors: John Trant, windsor (CA); Abhinandan Banerjee, windsor (CA); Kasra Razmkah, windsor (CA); Behzad Bolandi, windsor (CA); William Charles Hosie, windsor (CA); Mary Egbuta, windsor (CA)

(21) Appl. No.: 18/405,842

(22) Filed: Jan. 5, 2024

Related U.S. Application Data
(60) Provisional application No. 63/437,448, filed on Jan. 6, 2023.

(51) Int. Cl. A61K 9/107; A61K 9/00; A61K 31/137; A61K 31/355; A61K 31/4045; A61K 31/55; A61K 31/675; A61K 47/10; A61K 47/14

(52) U.S. Cl. CPC A61K 9/1075; A61K 9/0043; A61K 31/137; A61K 31/355; A61K 31/4045; A61K 31/55; A61K 31/675; A61K 47/10; A61K 47/14

(57) ABSTRACT

Disclosed is a process for the manufacture of the self-nanoemulsifying drug delivery systems (SNEDDS) of the present invention by a process that comprises: (a) combining (i) a therapeutic amount of one or more tryptamine-scaffold psychedelics, (ii) a triglyceride oil, (iii) a first surfactant, (iv) a second surfactant, and, optionally, (v) an anti-oxidant in a container, (b) adding a lower chain alcohol to said container while mixing the ingredients therein to form a pre-emulsion product, (c) optionally cooling said pre-emulsion product, and (d) diluting the pre-emulsion product with water to form a self-nanoemulsifying drug delivery system.

This application is related to U.S. Provisional patent application Ser. No. 63/437,448 that was filed on 6 Jan. 2023 and whose disclosure is incorporated herein by reference.

### FIELD OF THE INVENTION

The invention relates to formulations of tryptamine-scaffold psychedelic agents that are useful for therapy and/or nutrition and methods for their manufacture.

### BACKGROUND OF THE INVENTION

Many people worldwide are afflicted with psychological or mood disorders, such as depression, anxiety, compulsion, and post-traumatic stress disorders. Many of these conditions are believed to involve a person's serotonin system-including interactions between (A) the neurotransmitter serotonin (often abbreviated 5-HT) and (B) several different subtypes of serotonin neurotransmitter receptors found in the human body.

A variety of compositions are known to modulate activity at the serotonin receptors. A number of pharmaceuticals (antidepressants, serotonin reuptake inhibitors, selective serotonin reuptake inhibitors, etc.) have become available. Almost all these pharmaceuticals target neurotransmitters, e.g., serotonergic receptors, adrenergic receptors, dopaminergic receptors, etc., and in different ways. All ten of the leading pharmaceutical products for treating mood disorders (such as depression, obsessive compulsive disorder, and/or anxiety disorders) target serotonin pathways.

A composition including psilocybin (4-phosphoryloxy-N,N-dimethyltryptamine) or psilocin (4-hydroxy-N,N-dimethyltryptamine) in pure form or extracts from Psilocybe and psilocybin containing mushrooms combined with erinacines or hericenones, or extracts from Hericium mushroom species, and niacin (nicotinic acid or 3-pyridinecarboxylic acid, also known as vitamin B3), uniquely aids in repairing and improving neurologic functioning and signaling. Schartner et al. (2017) reported substantial increased global neural signal diversity in a psilocybin-human clinical study (Nature Scientific Reports, 7:46421). Additionally, niacin is known to be a neural anti-inflammatory, and, in itself, has been implicated in improving neural functioning. As niacin activates nerve endings, the inventor suggests that the addition of niacin contributes an added benefit by enhancing the neurogenic effects of psilocybin, psilocin, erinacines and hericenones by helping these nootropics cross the blood brain barrier, and migrate throughout the nervous systems, and to its end points. Moreover, niacin is a vasodilator improving blood flow in the brain by relaxing constricted blood vessels. This unique combination not only rebuilds myelin upon the axons, it also activates new astrocyte/astroglial cells and neuronalnodes of crossings such as the synaptic regions, particularly in the hippocampus. Other medicinal mushroom species also can be added, particularly species of Antrodia, Beauveria, Copelandia, Cordyceps, Ganoderma, Grifola, Inonotus, Isaria, Panaeolus, Phellinus, and other medicinal mushrooms and their mycelia whose unique neurogenerative properties may add benefits to this basic formulation. An excellent summary of the prior art related to the use of mushrooms as “brain foods” can be found in Phan et al. (2017), “Edible and Medicinal Mushrooms: Emerging Brain Food for the Mitigation of Neurodegenerative Diseases,” Journal of Medicinal Foods 20(1): 1-10. Lion's Mane (Hericium erinaceus), Bear's Head (H. coralloides), or CombTooth (H. ramosum) mushrooms and mycelium have also been well studied and reported to regenerate myelin on the axons of nerves. Two particular families of compositions are most noteworthy—erinacines and hericenones. Erinacines, including known erinacines A-K, P and Q, are cyanthane terpenes isolated from the mycelia of Hericium erinaceus that promote NGF (nerve growth factor) synthesis. Hericenones, including known hericenones C—H, are cyanthane terpenes located in both the mycelia and fruiting body of Hericium erinaceus that promote NGF synthesis. Friedman et al. (2015) summarizes these activities in “Chemistry, Nutrition, and Health-Promoting Properties of Hericium ennaceus (Lion's Mane) Mushroom Fruiting Bodies and Mycelia and Their Bioactive Compositions,” Journal of Agricultural and Food Chemistry 63: 7108-7123.

Although Phan et al. describes many species with potential neurogenerative properties, the psilocybin or psilocybian species (i.e., “psilocybin-containing”) are not mentioned, either alone or in combinations with the edible and medicinal mushroom species described by Phan. A good summary of the role of psilocybin in humans can be found in Passie et al. (2002), “The Pharmacology of Psilocybin,” Addiction Biology 7: 357-364. That psilocybin has neurogenerative properties was elucidated by Catlow et al. (2013), “Effects of psilocybin on hippocampal neurogenesis and extinction of trace fear conditioning,” Experimental Brain Research 228: 481-491. See also US Publication Number 2022/0313367. The disclosures of these publications are hereby incorporated by reference.

Both the medical establishment and conventional wisdom define psychedelic substances, (including those in in the triptan family, and including substances classified as 5-HT2A agonists), by their ability to determine certain alterations in consciousness, emotion, and cognition, including positive and negative psychotomimetic symptoms (e.g., psychedelic effects, psychedelic experience, psychotomimetic effects). These effects are known to laymen and doctors for their potential recreational misuse and to researchers in the psychiatric field for their potential therapeutic uses in psychiatry and research applications for the study of brain function. In the case of substances in the triptan family (a family of tryptamine—scaffold-based drugs), these psychedelic/psychotomimetic effects are thought to be primarily induced by agonist actions at the 5-HT 2A receptor in the 5-HT receptor family.

Psychedelic substances are presently under investigation for the treatment of several psychiatric diseases and symptoms, including depression, PTSD, OCD, addiction, end-stage-cancer-associated anxiety. The psychedelic experience, which includes positive and negative psychotomimetic effects induced by a serotonin agonist, is an integral part of the intended treatment. For therapeutic purposes, serotonin agonist psychedelic drugs are administered in a particular “setting” and preceded and followed by counseling and or psychotherapy and the whole session is supervised and closely monitored. The administration of the serotonin agonist at a dose that produces psychedelic and or psychotomimetic symptoms should be paired with ancillary therapies, which include a particular physical setting, in addition to pre, during and post drug administration counseling and/or psychotherapy (talk therapy) to achieve therapeutic efficacy for certain psychiatric disorders. The psychedelic experience (which includes alterations in consciousness, emotion, and cognition, and positive and negative psychotomimetic symptoms) is thus viewed by researchers and scientists as integral part of the potential therapeutic efficacy of psychedelic drugs. See US Publication No. 2022/0143051 which is hereby incorporated by reference.

The development of tryptamine-scaffold psychedelic agent pharmaceuticals and nutraceuticals face challenges because vital active product ingredients (APIs) that are typically derived from such mushrooms are difficult to administer reliably by direct ingestion. Even when “magic mushrooms” are properly identified, those mushrooms vary greatly in terms of the concentration of psilocybin, psilocin, and other (often overlooked) active ingredients. Accordingly, administering a specific composition or a particular dose using mushrooms is not reliable because of the variability in the chemical composition of mushrooms even in the same species.

Some of the major challenges to be overcome to create stable, robust, reproducible, and biologically active products include:
a. Lipophilicity and low water solubility of API,
b. Limited bioaccessibility of API,
c. Extensive degradation of API during formulation, storage, or in vivo owing to metabolic pathways, and
d. Inability of the API to cross the blood-brain barrier if psychoactivity is to be demonstrated.

It would be desirable to have a composition and method for administering psilocin and/or psylocibin that would provide a consistent, therapeutically useful dose.

Phytochemical active pharmaceutical ingredients (APIs), especially tryptamine-scaffold psychedelics such as psilocybin, mescaline, psilocin, ibogaine and bufotenin, are being studied for their use in the treatment of major depressive disorder (MDD), substance use disorder (SUD), and neurodegenerative disorders, neurological disorders, and inflammatory disorders. Formulation and delivery challenges associated with even the serum-soluble tryptamine-scaffold APIs and their prodrugs—such as stress-induced degradation, limited bioavailability, and significant first-pass metabolism—have combined to make it difficult to identify functional formulations.

Bufotenine (5-Hydroxy-N,N-dimethyltryptamine) is a naturally-occurring tryptamine derivative and constitutional isomer of psilocin found in a range of fungal, plant, and animal species such as seeds of the Anadenanthera genus, mushrooms of the Amanita genus, and in the venom and eggs of toads in the Bufo genus. They have been used for millennia for their hallucinogenic effects.

Seeds of the Anadenanthera colubrina and Anadenanthera peregrina were smoked by indigenous peoples of the northern regions of Argentina up to 4000 years ago. These seeds (containing up to 15 wt % of bufotenine) were dried, roasted and ground to produce a powdered preparation known as a snuff (“hataj”, “cohoba”, “yopo”) for insufflation and smoking in pipes. Modern literature tells us that intranasal, intravenous, and smoking (inhalation) are the common forms of administration due to rapid metabolization of bufotenine during oral administration. Inhalation of bufotenin (2-8 mg) produces effects within 4-5 mins that last for up to one hour. Bufotenin is not under international control and has experienced a recent resurgence in interest, along with other psychedelic APIs, for the therapeutic treatment of MDD, SUD, and various neurodegenerative disorders.

Currently, it is statistically significant that people who are prescribed anti-depressants are subjected to difficult and sometimes debilitating side effects that affect their daily lives. Depression is one of the largest epidemics in the world, which suggests that other classes of therapeutics must be investigated to provide effective therapies for its treatment. This has led to renewed interest in cannabis and entheogen research.

The fundamental idea behind the current work is a “Trojan Horse” delivery system that uses some type of encapsulating technology to overcome the variable solubility profiles, bio-accessibility, and susceptibility to oxidative degradation of psilocin APIs. Such encapsulating delivery systems carry the API payload to intracellular destinations that are otherwise inaccessible to the unformulated APIs.

There are very few examples of formulations of tryptamine scaffold psychedelic APIs in the literature. However, these formulations could readily be consumed in a clinical setting or as a prescription medication without the need for special storage or in situ formulation by a chemist.

It would be desirable to have an effective formulation for the delivery of tryptamine-scaffold psychedelics (such as psilocybin, mescaline, psilocin, ibogaine and bufotenin) that would be bioavailable and bioeffective in therapeutically effective doses.

Most formulation approaches to improve bioavailability of water insoluble, highly lipophilic drugs are based on either particle size reduction technologies (e.g. micronization or nano-particle generation) to increase drug dissolution rate and/or achieve transient solubilization, or technologies to achieve a sustained solubilization of the drug, such as complexation, or use of lipid-based delivery systems. The particle size reduction technologies often fail to overcome bioavailability limitations. See U.S. Pat. No. 11,617,758.

A widely utilized approach to achieve sustained solubilization and overcome poor fasted state bioavailability of lipophilic drugs is to utilize solutions in lipid vehicles containing surfactants that constitute a self-emulsifying drug delivery system (SEDDS), to effect spontaneous emulsification upon contact of the lipid with fluids in the GI tract. If micro-emulsions or nano-emulsions are formed, these are referred to as self-microemulsifying drug delivery systems (SMEDDS) or SNEDDS.

SEDDS produce opaque, white emulsions with lipid droplet sizes of >200 nm, while SMEDDS or SNEDDS form transparent or translucent microemulsions with droplet size of less than 200 nm.

These emulsions are isotropic mixtures of (a) at least one drug, (b) an organic vehicle, (c) a surfactant, and (d) a co-solvent. These formulations rapidly form relatively stable oil-in-water (o/w) emulsions where the drug is contained in micron-size or nanometer-size droplets for SMEDDS and SNEDDS, respectively, upon aqueous dilution in gastrointestinal fluids.

SNEDDS show high drug solubilizing capacity and enhancement in both rate and extent of absorption by the lymphatic uptake. Moreover, it is possible to form blends that are composed of several excipients, such as pure triglycerides or mixtures of mono-, di- and triglycerides.

Orally administrated SNEDDS widen the accessibility of lipidic excipients with to offer flexibility of function with respect to improving bioavailability of drugs by manipulating their release profiles and protecting them from enzymatic and/or chemical hydrolysis while facilitating their passage in the gastrointestinal tract until their intestinal absorption. SNEDDS may also demonstrate cell-penetrating properties. SNEDDS can be simply manufactured using hot or cold mixing, at low cost.

It would be desirable to have an effective process to make a stable, effective, self-nanoemulsifying drug delivery composition that could be used with tryptamine-scaffold psychedelics.

It would be further desirable to have a SNEDDS process to make a composition comprising tryptamine-scaffold psychedelics in therapeutically effective concentrations to produce a therapeutically effective composition for the treatment of a human or mammal patient.

### SUMMARY OF THE INVENTION

It is an object of the invention to provide a composition and method for its administration to a patient having a therapeutic need that would provide a consistent, therapeutically useful dose of a tryptamine-scaffold psychedelic.

It is also an objective of the invention to provide a composition and method of administration that would delivery a therapeutically effective amount of a tryptamine-scaffold psychedelic to patient in need of same that would provide a bioavailable, bioeffective dose in therapeutically effective dosages.

It is further an objective of the invention to provide an effective process to make a stable, effective, self-nanoemulsifying drug delivery composition that could be used with tryptamine-scaffold psychedelics.

Additionally, it is an object of the invention to provide a SNEDDS process to make a composition comprising tryptamine-scaffold psychedelics in therapeutically effective concentrations to produce a therapeutically effective composition for the treatment of a human or mammal patient.

In accordance with these and other objects of the invention that will become apparent from the description herein, compositions according to the invention are in the form of an oil-in-water nano-emulsion that comprises: (a) a therapeutic amount of one or more tryptamine-scaffold psychedelics, (b) a triglyceride oil, (c) a first hydrophilic surfactant, (d) a second surfactant comprising polyethylene glycol 660 12-hydoxystearate, and, optionally, (e) an anti-oxidant comprising Vitamin E.

The invention also contemplates a process for the manufacture of the SNEDDS of the present invention by a process that comprises: (a) combining (i) a therapeutic amount of one or more tryptamine-scaffold psychedelics, (ii) a triglyceride oil, (iii) a first surfactant, (iv) a second surfactant, and, optionally, (v) an anti-oxidant in a container, (b) adding a lower chain alcohol to said container while mixing the ingredients therein to form a pre-emulsion product, (c) optionally cooling said pre-emulsion product, and (d) diluting the pre-emulsion product with water to form a self-nanoemulsifying drug delivery system.

The nano-emulsion form of the present invention provides a stable formulation that is well-suited for delivery of the one or more tryptamine-scaffold psychedelics via nasal spray thereby protecting the emulsion and any active ingredient from the harsh conditions associated with oral ingestion and digestion. The small average droplet size of the nano-emulsion and ability of the active ingredient to cross the blood-brain barrier present unique opportunities for compositions that can deliver therapeutic benefits without the limitations encountered by prior efforts.

### BRIEF DESCRIPTION OF THE DRAWINGS

FIG. 1 is a graph showing the average droplet size and polydispersity index (PDI) for Formula A upon exposure to different pH values. Error bars represent N=3 replicate experiments.

FIG. 2 similarly shows the average droplet size and polydispersity index (PDI) for Formula B upon exposure to different pH values. Error bars also represent N=3 replicate experiments.

### DETAILED DESCRIPTION

Disclosed herein is a design of a stable, self-emulsifying, drug delivery system exhibiting nanometer-sized droplets containing (a) one or more tryptamine-scaffold psychedelic agents in the emulsified droplets. Each of these is a lipophilic drug that is poorly soluble in water and well suited for SNEDDS delivery. Preferred tryptamine-scaffold psychedelic agents comprise psilocybin, mescaline, psilocin, ibogaine, and/or bufotenin in an amount within the range of 1-80 wt %, preferably 15-30 wt % based on total weight of the SNEDDS. When used in a liquid form, a suitable amount of the tryptamine-scaffold psychedelic agents is an amount within the range from about 0.1-1000 mg/ml, preferably an amount within the range of 0.5-500 mg/ml, and more preferably an amount within the range of 1-100 mg/ml.

The composition of the present invention also includes (b) an organic vehicle comprising a triglyceride oil, (c) a first surfactant, (d) a second surfactant, and, optionally, (e) an anti-oxidant.

Suitable organic vehicles include medium-chain and long-chain triglyceride oils. The triglyceride oil is generally used in an amount within the range from about 5-50 wt %, preferably an amount within the range of 10-40 wt %, and even more preferably an amount within the range of 20-35 wt % based on total SNEDDS weight.

Suitable first surfactant is generally a hydrophilic surfactant that is selected from p-isononylphenoxypolyglycidol, PEG laurate, Tween 20, Tween 40, Tween 60, Tween 80 (polyoxyethylene (80) sorbitan monooleate), PEG oleate, PEG stearate, PEG glyceryl laurate, PEG glyceryl oleate, PEG glyceryl stearate, polyglyceryl laurate, plyglyceryl oleate, polyglyceryl myristate, polyglyceryl palmitate, polyglyceryl-6 laurate, plyglyceryl-6 oleate, polyglyceryl-6 myristate, polyglyceryl-6 palmitate, polyglyceryl-10 laurate, plyglyceryl-10 oleate, polyglyceryl-10 myristate, polyglyceryl-10 palmitate PEG sorbitan monolaurate, PEG sorbitan monolaurate, PEG sorbitan monooleate, PEG sorbitan stearate, PEG oleyl ether, PEG laurayl ether, octoxynol, monoxynol, tyloxapol, sucrose monopalmitate, sucrose monolaurate, decanoyl-N-methylglucamide, n-decyl-β-D-glucopyranoside, n-decyl-β-D-maltopyranoside, n-dodecyl-β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-noyl-β-D-glucopyranoside, octanoyl-N-methylglucamide, n-octyl-β-D-glucopyranoside, octyl-β-D-thioglucopyranoside; cystine, tyrosine, tryptophan, leucine, isoleucine, phenylalanine, asparagine, aspartic acid, glutamic acid, and methionine; acetic anhydride, benzoic anhydride, ascorbic acid, 2-pyrrolidone-5-carboxylic acid, sodium pyrrolidone carboxylate, ethylenediaminetetraacetic dianhydride, maleic and anhydride, succinic anhydride, diglycolic anhydride, glutaric anhydride, acetiamine, benfotiamine, pantothenic acid; cetotiamine; cycothiamine, dexpanthenol, niacinamide, nicotinic acid, pyridoxal 5-phosphate, nicotinamide ascorbate, riboflavin, riboflavin phosphate, thiamine, folic acid, menadiol diphosphate, menadione sodium bisulfite, menadoxime, vitamin B12, vitamin K5, vitamin K6, vitamin K6, and vitamin U; albumin, immunoglobulins, caseins, hemoglobins, lysozymes, immunoglobins, a-2-macroglobulin, fibronectins, vitronectins, firbinogens, lipases, benzalkonium chloride, benzethonium chloride, docecyl trimethyl ammonium bromide, sodium docecylsulfates, dialkyl methylbenzyl ammonium chloride, and dialkylesters of sodium sulfonsuccinic acid, L-ascorbic acid and its salt, D-glucoascorbic acid and its salt, tromethamine, triethanolamine, diethanolamine, meglumine, glucamine, amine alcohols, glucoheptonic acid, glucomic acid, hydroxyl ketone, hydroxyl lactone, gluconolactone, glucoheptonolactone, glucooctanoic lactone, gulonic acid lactone, mannoic lactone, ribonic acid lactone, lactobionic acid, glucosamine, glutamic acid, benzyl alcohol, benzoic acid, hydroxybenzoic acid, propyl 4-hydroxybenzoate, lysine acetate salt, gentisic acid, lactobionic acid, lactitol, sinapic acid, vanillic acid, vanillin, methyl paraben, propyl paraben, sorbitol, xylitol, cyclodextrin, (2-hydroxypropyl)-cyclodextrin, acetaminophen, ibuprofen, retinoic acid, lysine acetate, gentisic acid, catechin, catechin gallate, tiletamine, ketamine, propofol, lactic acids, acetic acid, salts of any organic acid and organic amine, polyglycidol, glycerol, multiglycerols, galactitol, di(ethylene glycol), tri(ethylene glycol), tetra(ethylene glycol), penta(ethylene glycol), poly(ethylene glycol) oligomers, di(propylene glycol), tri(propylene glycol), tetra(propylene glycol, and penta(propylene glycol), poly(propylene glycol) oligomers, a block copolymer of polyethylene glycol and polypropylene glycol, and derivatives and combinations thereof. An especially preferred lipophilic surfactant is polyoxyethylene (80) sorbitan monooleate.

A preferred second surfactant comprises polyethylene glycol 660 12-hydoxystearate, CAS No. 70142-34-6. This surfactant is available from BASF Corporation in Florham Park, NK under the name KOLLIPHOR HS 15 and Kolliphor® HS 15 is a nonionic solubilizer and emulsifying agent obtained by reacting 15 moles of ethylene oxide with 1 mole of 12-hydroxy stearic acid.

Suitable anti-oxidants used in the SNEDDS of the invention include those related to Vitamin E. “Vitamin E” refers to a group of compounds that include both tocopherols and tocotrienols. The α-tocopherol compound is a bio-active form of vitamin E and is a fat-soluble antioxidant that interrupts the propagation of reactive oxygen species that spread through biological membranes. Alpha-tocopherol functions within the glutathione peroxidase pathway in-vivo and protects compounds and biological membranes from oxidation. This is because α-tocopherol reactions with lipid radicals produced in the lipid peroxidation chain reaction or through a fat when its lipid content undergoes oxidation by reacting with more-reactive lipid radicals to form more stable products.

The total amounts of first and second surfactant, combined, are generally within a range from about 5-50 wt %, preferably an amount within the range of 10-40 wt %, and more preferably an amount within the range from about 15-35 wt % of the total SNEDDS weight.

Suitable transient lower alcohols for use in making the present invention include the C2-C4 alcohols and their isomers, preferably ethanol or a propanol such as 2-propanol.

The general method of treatment comprises administering to a human or mammal subject in need thereof a therapeutically-effective amount of an acceptable psychedelic analogue in one or more pharmaceutically acceptable carriers or excipients.

The administered composition is formulated for oral, sublingual, intranasal, pulmonary administration, buccal, sublingual, rectal, transdermal, transmucosal, epidural, intrathecal, intraocular topical, creams, lotions, gels and eye drops using one or more excipients that are traditionally used in such formulations. A preferred form of delivery is by way of a metered spray of a volume of liquid SNEDDS into a patient's nasal cavity.

In another aspect, the invention comprises a method of treating a mental disorder, comprising the step of administering an effective amount of a ligand described herein. In some embodiments, the mental disorder is a depressive condition, including unipolar and bipolar depressive conditions, such as but not limited to depression, depression from generalized anxiety, major depression, treatment resistant depression and postpartum depression.

The invention provides for the treatment and/or prevention of psychiatric disorders, and/or neurological disorders, and/or degenerative disorders, and/or inflammatory disorders. In another aspect, the invention relates to the use of a composition described herein to treat a mental disorder, or in the manufacture of a medicament for treating a mental disorder, such as depression.

As used herein, the term “neurological disorders” refers to any structural, biochemical and/or electrical abnormalities in the brain, spinal cord or other nerves and includes neurodevelopment and neurodegenerative diseases that may benefit from of neural plasticity modulation. In a preferred embodiment, the term “neurological disorder” refers to one or more disorders selected from the following acquired brain injury, ataxia brain tumor, dementia, dystonia epilepsy, temporal lobe epilepsy, pain associated with neurological disorders, headache disorders, functional and dissociative neurological symptoms, neuroinfections, meningitis, disorders associated with malnutrition, motor neuron disease, multi-system atrophy, multiple sclerosis, amyotrophic lateral sclerosis, mesial temporal lobe hippocampal sclerosis, muscular dystrophy, myalgic encephalomyelitis, Parkinson's disease, progressive supranuclear palsy, cerebral palsy, Huntington's disease, Alzheimer's disease, frontal lobe dementia, vascular dementia, dementia with Lewy bodies, mild cognitive impairment (MCI) associated with aging and chronic disease and its treatment, including chemotherapy, immunotherapy and radiotherapy, mild corticobasal degeneration, disorders associated with accumulation of beta amyloid, and/or with the accumulation or distruption of tau protein and its metabolites. Lyme encephalopathy, toxic encephalopathy, cognitive decline associated with aging, spinabifida, hydrocephalus, spinal injury, stroke, Tourette syndrome, and transverse myelitis, corticobasal degeneration, supranuclear palsy, epilepsy; nervous system trauma, nervous system infections, nervous system inflammation, including inflammation from autoimmune disorders, including NMDAR encephalitis, and cytopathology from toxins, (including microbial toxins, heavy metals, and pesticides etc.), stroke, multiple sclerosis, Huntington's disease, mitochondrial disorders, Fragile X syndrome, Angelman syndrome, hereditary ataxias, neuro-otological and eye movement disorders, amyotrophic lateral sclerosis, tardive dyskinesias (TD), hyperkinetic disorders; attention deficit hyperactivity disorder and attention deficit disorders; restless leg syndrome, autism spectrum disorders, tuberous sclerosis, Rett syndrome, cerebral palsy, disorders of the reward system including eating disorders [including anorexia nervosa (“AN”) and bulimia nervosa (“BN”), and binge eating disorder (“BED”), trichotillomania, dermotillomania, nail biting, migraine, fibromyalgia, and peripheral neuropathy of any etiology. Symptoms or manifestations of nervous system disorders that may be treated or prevented by neuroplastogen substances and drugs include, a decline, impairment, or abnormality in cognitive abilities including executive function, attention, cognitive speed, memory, language functions (speech, comprehension, reading and writing), orientation in space and time, praxis, ability to perform actions, ability to recognize faces or objects, concentration, and alertness; abnormal movements including akathisia, bradykinesia, tics, myoclonus, dyskinesias, including dyskinesias relate to Huntington's disease, levodopa induced dyskinesias and neuroleptic induced dyskinesias, dystonias, tremors, including essential tremor, and restless leg syndrome; parasomnias, insomnia, disturbed sleep pattern; psychosis; delirium; agitation; headache; motor weakness, spasticity, impaired physical endurance; sensory impairment, including impairment of vision and visual field defects, smell, taste, hearing and balance, and dysesthesias; dysautonomia; and ataxia, impairment of balance or coordination, tinnitus, neuro-otological and eye movement impairments, neurological symptoms of alcohol withdrawal, including delirium, headache, tremors, hallucinations, hypertension.

The term “degenerative disorders” refers to one or more disorders selected from the following degenerative disorders, neurodegenerative diseases of the retina like glaucoma, diabetic retinopathy and age-related macular degeneration, retinitis pigmentosa, Usher disease and Bardet-Biedl syndrome, motor neuron disease, prion disease, spinocerebelluar ataxia and apathy syndrome.

The term “inflammatory disorders” refers to one or more disorders selected from the following inflammatory disorders, of atherosclerosis, asthma, rheumatoid arthritis, psoriasis, type II diabetes, irritable bowel syndrome, Crohn's disease, septicemia, depression, schizophrenia, multiple sclerosis, conjunctivitis, Alzheimer's disease, chronic obstructive pulmonary disease, neuro-inflammation, metabolic syndrome, impaired glucose tolerance, non-alcoholic fatty liver disease (NAFLD), (NAFL) and their complications, nonalcoholic steatohepatitis (NASH) and conjunctivitis.

The general method of treatment comprises administering to a human or mammal subject in need thereof a therapeutically-effective amount of an acceptable psychedelic analogue in one or more pharmaceutically acceptable carriers or excipients.

The term “treating”, “treat” or “treatment” as used herein embraces both preventative, i.e., prophylactic, and palliative treatment, i.e., relieve, alleviate, or slow the progression of the patient's disease, disorder, or condition.

As used herein, “psychedelic state” is an altered state of consciousness experienced by a person, which may include intensified sensory perception, perceptual distortion or hallucinations, and/or feelings of euphoria or despair. Psychedelic states have been described as resulting from psychedelic drugs such as DMT (dimethyltryptamine), LSD, mescaline or psilocybin. Other known psychedelic drugs include the 4-hydroxy analogs of N-Methyl-N-isopropyltryptamine (MiPT) and N,N-diisopropyltryptamine (DiPT).

The term “psychiatric disorders” refers to one or more disorders selected from the following psychiatric disease as defined as defined by DMS5 and ICD11 that may benefit from modulation of neural plasticity, including Schizophrenia spectrum and other psychotic disorders, Bipolar and related disorders, Depressive disorders, COVID Depressive disorder, generalized anxiety disorders, Obsessive-compulsive and related disorders, Trauma- and stressor-related disorders, dissociative disorders, somatic symptom and related disorders, feeding and eating disorders, elimination disorders, sleep-wake disorders, sexual disruptive, impulse-control, and conduct disorders, substance-related and addictive disorders, panic disorder, agoraphobia, social anxiety disorder, phobias, posttraumatic stress disorder, obsessive compulsive disorder, generalized anxiety disorder, anorexia nervosa, binge eating disorder, bulimia nervosa, psychosis, schizophrenia, substance addiction, personality disorders, neurocognitive disorders, personality disorders, paraphilic disorders and for the reduction of suicidal ideation in a patient suffering from a life-threatening disease.

### Formulations and Compositions

The invention also provides pharmaceutically acceptable compositions which comprise a therapeutically effective amount of one or more of the compositions described herein, formulated together with one or more pharmaceutically acceptable carriers (additives) and/or diluents, and optionally, one or more additional therapeutic agents. While it is possible for a composition described herein to be administered alone, it is preferable to administer the composition as a pharmaceutical composition.

The term “pharmaceutical composition” means a composition comprising a composition of the invention in combination with at least one additional pharmaceutically acceptable carrier.

A “pharmaceutically acceptable carrier” refers to media generally accepted in the art for the delivery of biologically active agents to animals, in particular, mammals, including, i.e., adjuvant, excipient or vehicle, such as diluents, osmotic complement, preserving agents, fillers, flow regulating agents, disintegrating agents, wetting agents, emulsifying agents, suspending agents, sweetening agents, flavoring agents, perfuming agents, antibacterial agents, antifungal agents, lubricating agents, polymers, solubilizing agents, stabilizers, antioxidants and dispensing agents, depending on the nature of the mode of administration and dosage forms. Each carrier must be “acceptable” in the sense of being compatible with the other ingredients of the formulation and not injurious to the patient.

As used herein, “oral” administration includes swallowing for ingestion in the stomach or gut, and further includes lingual, sublingual, buccal and oropharyngeal administration. The compositions of this invention can be administered for any of the uses or methods described herein by any suitable means, for example, orally, such as tablets, capsules (each of which may include sustained release or timed release formulations), pills, powders, granules, elixirs, suspensions (including nano suspensions, micro suspensions, spray-dried dispersions), syrups, and emulsions; sublingually (e.g. as thin films, effervescent tablets or tablets that dissolve spontaneously under the tongue); parenterally, such as by subcutaneous, intravenous, intramuscular injection, or infusion techniques (e.g., as sterile injectable aqueous or non-aqueous solutions or suspensions); nasally, including administration to the nasal membranes, such as by inhalation spray; or rectally such as in the form of suppositories.

The dosage regimen for the compositions described herein will, of course, vary depending upon known factors, such as the pharmacokinetic and pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient; and, the effect desired. The selected dosage level may also depend on the additional factors including the activity of the particular compositions and pharmaceutical compositions described herein, whether an ester, salt or amide substituent is of the composition is used, the time of administration, the rate of excretion or metabolism of the particular composition being employed, the rate and extent of absorption, the duration of the treatment, other drugs that may be administered to the patient, compositions and/or materials used in combination with the particular composition employed and like factors well known in the medical arts.

Generally, the dosage of the prodrug for a therapy session, when used for the indicated effects, will range between about 0.001 to about 500 mg per dose, preferably between about 0.01 to about 200 mg per dose, and most preferably between about 0.1 to about 50 mg per dose, such as 10, 20, 30, 40, 50, 100 or 200 mg. Intravenously, the most preferred doses will range from about 0.01 to about 10 mg/kg/minute during a constant rate infusion.

Compositions of the present invention may be administered in a single daily dose, or the total daily dosage may be administered in multiple divided doses, such as two, three, or four times daily. Alternatively, the doses may be provided on a weekly, biweekly, or monthly basis. In a preferred embodiment, only one or two doses are required for an anti-depressant effect that may extend for 1, 2, 3 or 6 months, or more.

For tablet dosage forms, depending on dose, the composition of the present invention may make up from 1 wt % to 80 wt % of the dosage form, more typically from 5 wt % to 60 wt % of the dosage form.

In addition to the present composition, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste masking agents.

Exemplary tablets contain up to about 80 wt % of the present composition, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet, dry, or melt granulated, melt congealed, or extruded before tableting. The final formulation may include one or more layers and may be coated or uncoated; or encapsulated.

A typical capsule for oral administration contains at least one of the formulations of the present invention (e.g. 25 mg), lactose (e.g. 75 mg), and magnesium stearate (e.g. 15 mg). The mixture is passed through a 60 mesh sieve and packed into a No. 1 gelatin capsule.

Liquid formulations include suspensions, solutions, syrups, and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Liquid formulations may also be administered in the form of a nasal spray that provides direct contact with mucosal membranes in relatively close proximity to the blood-brain barrier. A preferred mechanism for delivery is a nasal spray that delivers a metered amount with each pump so the volume of sprayed nano-emulsion is substantially consistent from dose to dose.

Compositions of the invention may be combined with soluble macromolecular entities, such as cyclodextrin and suitable derivatives thereof or polyethylene glycol containing polymers, in order to improve their solubility, dissolution rate, taste masking, bioavailability and/or stability for use in any of the aforementioned modes of administration.

Drug cyclodextrin complexes, for example, are found to be generally useful for most dosage forms and administration routes. Both inclusion and non-inclusion complexes may be used. As an alternative to direct complexation with the drug, cyclodextrin may be used as an auxiliary additive, i.e. as a carrier, diluent, or solubilizer. The materials most commonly used for these purposes are alpha, beta and gamma cyclodextrins, examples of which may be found in PCT Publication Nos. WO 91/11172, WO 94/02518 and WO 98/55148, the disclosures of which are incorporated herein by reference in their entireties.

Regardless of the route of administration selected, the compositions of the present invention, which may be used in a suitable hydrated form, and/or the pharmaceutical compositions of the present invention, are formulated into pharmaceutically acceptable dosage forms by conventional methods known to those of skill in the art. Actual dosage levels of the active ingredients in the pharmaceutical compositions of this invention may be varied so as to obtain an amount of the active ingredient which is effective to achieve the desired therapeutic response for a particular patient, composition, and mode of administration.

A physician or veterinarian having ordinary skill in the art can readily determine and prescribe the effective amount of the pharmaceutical composition required. For example, the physician or veterinarian could start doses of the compositions of the invention employed in the pharmaceutical composition at levels lower than that required to achieve the desired therapeutic effect and gradually increase the dosage until the desired effect is achieved.

In general, a suitable daily dose of a composition of the invention will be an amount of the composition which is the lowest dose effective to produce a therapeutic effect. Such an effective dose will generally depend upon the factors described above.

As used herein, a “therapeutically effective amount” refers to that amount of a composition being administered which will relieve to some extent one or more of the symptoms of the disorder being treated. In reference to the treatment of depression, a therapeutically effective amount refers to that amount which has the effect of reducing the severity of depression. Depression severity may be assessed using well-known structured assessment tools such as Structured Clinical Interview for DSM-5 (SCID-5) and the GRID-Hamilton Depression Rating Scale (GRID-HAMD). A therapeutically effective amount may be less than that required for a psychedelic state.

An effective dosage can be administered in one or more administrations. For the purposes of this invention, an effective dosage of drug, composition, or pharmaceutical composition is an amount sufficient to accomplish prophylactic or therapeutic treatment either directly or indirectly. As is understood in the clinical context, an effective dosage of drug, composition or pharmaceutical composition may or may not be achieved in conjunction with another therapy, drug, composition, or pharmaceutical composition.

### Therapeutic Methods and Uses

Treatment with the novel prodrugs of the present invention may substantially alleviate clinical or subclinical depression and may avoid relapse, particularly if used in combination with psychotherapy for the treatment of depression. It is known that administration of an effective dose of psilocybin produced rapid and large reductions in depressive symptoms, and many subjects achieve remission through a four-week follow up (Davis et. al.) Without restriction to a theory, it is believed that the psychedelic state is associated with the beneficial effects, however, some compositions which are 5HT2AR agonists may provide the desired therapeutic effect without the psychedelic state. One aspect of the invention comprises prodrugs of those 5HT2AR agonists which do provide a beneficial therapeutic state.

In general, the present invention includes the use of a composition of the present invention herein, to treat any disease or disorder which may be alleviated by a 5HT2AR agonist, or the use of a composition of the present invention herein to manufacture a medicament to treat any disease or disorder which may be alleviated by a 5HT2AR agonist, or a method of treating any disease or disorder which may be alleviated by a 5HT2AR agonist.

In some embodiments, the invention may comprise the use of the compositions of the present invention to treat mental disorders. In some embodiments, the invention may comprise the use of the compositions of the present invention to treat depression, and particularly drug resistant depression. Other conditions that may be treated include: anxiety disorders, including anxiety in advanced stage illness e.g. cancer as well as generalized anxiety disorder, depression including major depressive disorder, postpartum depression, cluster headaches, obsessive compulsive disorder, personality disorders including conduct disorder, drug disorders including: alcohol dependence, nicotine dependence, opioid dependence, cocaine dependence and other addictions including gambling disorder, eating disorder and body dysmorphic disorder, chronic pain, or chronic fatigue.

In some embodiments, the invention may comprise a method of treating mental disorders comprising administering to a subject in need thereof a therapeutically effective amount of a composition of the present invention. In one embodiment, there is provided a method of treating depression by administering to a subject in need thereof therapeutically effective amount of a composition of the present invention. The depression effects may be drug-resistant depression or major depressive disorder.

For example, a patient diagnosed with depression may be screened prior to treatment, and then prepared for a dosing session by a trained psychotherapist. Within a dosing session, a composition of the present invention may be administered by injection of a sterile solution at a rate of 0.01-0.3 mg/kg to the patient. The patient is preferably seated for the duration of the session while being blindfolded. For safety, a trained health care professional may monitor the patient throughout the dosing session, which may last up to 12 hours. In some cases, music may be played for the patient. When the health care professional can determine that the drug substance has cleared, the psychotherapist may assist the patient with any questions relating to the psychedelic experience, and then the patient may be discharged.

To further alleviate any anxiety that may occur relative to therapy, the physician may prefer to divide the therapeutic dose and thereby reduce the initial onset of psychoactivity before applying the full complement of the dosage to achieve the full effect.

In some embodiments, treatment with a composition of the present invention may be combined with concomitant treatment with another anti-depressant drugs, either concurrently or consecutively. In preferred embodiments, treatment with a composition of the present invention is combined with psychotherapy, which may be applied prior to or after treatment. If prior to, the session may focus the patient on the intent of treatment. If after, psychotherapy is preferably performed within 48 hours of the dosing session to help the patient integrate any feelings, emotions, visions or thoughts that may have occurred during the session, as well as to allow the psychotherapist may offer advice on how best to change thinking or behavior patterns so as to improve anti-depression outcomes. Psychotherapy may continue as needed after the dosing session, for example, up to an additional 3 months, to help the patient integrate any experiences or learnings that occurred to the patient during the dosing session.

### EXAMPLES

Our testing was done with bufotenin (Bf), a regioisomer of psilocin that is not regulated as a controlled substance, but which behaves in formulation in ways that are similar to controlled tryptamine-scaffold psychedelic drugs.

Described herein are studies that were performed to develop an encapsulating nano-emulsion composition and its method of manufacture for tryptamine-scaffold psychedelic agents. The experimental design provided multiple compositions of different ratios of oil, surfactant, and co-surfactant, of which two compositions were identified that exhibited unusually good performance metrics. These compositions were prepared as pre-emulsion compositions that formed oil-in-water (o/w) nano-emulsions with mean droplet sizes under 100 nm upon contact with water or pH 7 buffer. The nano-emulsions showed enhanced colloidal stability under physical and chemical stressors, as well as upon storage. Formulated bufotenin showed higher chemical stability when compared to a solution of pure bufotenin dissolved in ethanol in the presence of stressors.

Accelerated oxidation stress tests were performed to confirm the augmented stability of the nano-formulated bufotenin within the NEs. Flow cytometry and fluorescence imaging established decidedly that cellular uptake of formulated bufotenin is order of magnitude higher than that of bufotenin dissolved in biological buffers. To the best of our knowledge, this is the first reported study of a tryptamine-scaffold API encapsulated in a self-emulsifying delivery system having nanometer-sized droplets.

The emulsions produced in our examples show enhanced stability under physical and chemical duress, as well as upon storage (≤3% API loss upon 3 weeks storage at 4° C.; ≤6% API loss upon 72 hours ambient storage in direct sunlight).

The preferred formulations offer enhanced protection to the API under conditions of simulated oxidative stress (˜30% bufotenin loss; heat at 37° C. for 8 h) in comparison with unformulated bufotenin dissolved in ethanol (≥50% loss).

Cell uptake studies including flow cytometry and fluorescence microscopy of glioma (brain) cells exposed to formulated vs. unformulated bufotenin confirm about 100% uptake of formulated bufotenin by the cells. In contrast, no unformulated bufotenin penetrated the glioma cells.

Cytotoxicity assays on four cell lines reveal negligible toxicity of formulated bufotenin at therapeutic concentrations. The self-emulsified drug delivery systems described in this study solidify at about 10° C. and can be formed into capsules or pellets which are easy to dispense.

As can be expected, selection of excipients and titration of their respective ratios are important to the final composition. For our tested systems, we used medium chain trigyceride (MCT) oil as the lipid, ethanol as the transient organic medium, and polyoxyethylene (80) sorbitan monooleate as the hydrophilic co-surfactant.

For our primary emulsifier, however, the following properties were desired: (i) low histamine release to prevent allergic reactions; (ii) thermal stability, suitable for sterilization and other heating events during creation of the formulation; (iii) lower hemolytic activity; and (iv) monographed in Ph. Eur. and USP-NF, as a valid pharma-grade additive. Solubilizers currently used for parenteral administration such as Cremophor EL and polysorbate 80 have been implicated in clinically important adverse effects such as hypersensitivity reactions and a highly increased systemic drug exposure along with a reduced cellular uptake. To overcome these problems, we used nonionic surfactant called Kolliphor HS 15 (Polyethylene glycol 660 12-hydoxystearate) available from BASF in Florham Park, New Jersey) and believed to have the following chemical structure:

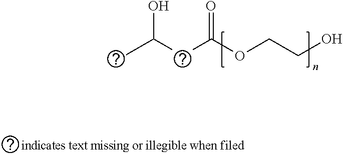

Kolliphor HS15 mainly contains polyoxyl 15 hydroxystearate obtained from the reaction of about 15 mol of ethylene oxide with 1 mol of 12-hydroxystearic acid. Polyoxyl 15 hydroxystearate has about 70% of polyglycol mono- and di-esters (lipophilic) and about 30% of free polyethylene glycol (PEG, hydrophilic). Kolliphor HS15 was found to enable poorly soluble drugs, such as the tryptamine-scaffold psychedelic agents of the present invention, to exhibit enhanced solubility and to partially inhibit the P-glycoprotein efflux mechanism. Thus, this emulsifier greatly increases the transportation of drug in the systemic circulation for poorly soluble drugs and further boosts their bioavailability.

The initial phase of the study focused on optimizing the relative amounts of various components of the emulsion for enhanced colloidal properties. The results of these optimization studies led us to two representative formulations, formulation A (Kolliphor-Bf emulsion) and formulation B (formulation A with the lipidic antioxidant α-tocopherol as an additive) in the form of pre-emulsion products. The pre-emulsion products were then mixed with water to form emulsions that were further diluted (if needed) with water to form nano-emulsions of the desired concentration. The resulting nano-emulsions were then characterized by dynamic light scattering to determine the average diameter of micellar droplets as well as to measure changes in polydispersity index and surface charge (ζ-potential). Emulsion droplets were imaged using a scanning electron microscope after appropriate sample preparation.

The nano-emulsions were stress tested under different chemical and physical environments mimicking commercial production and storage conditions to evaluate its long-term stability. The average droplet sizes, polydispersibility index, and zeta potentials for our nano-emulsions were promising under this stress testing.

Biological assays were used to evaluate the effect of our nano-emulsions on cells. Cytotoxicity was evaluated using normal and cancer cells for both formulations A and B. Cellular uptake studies confirmed that formulations A and B both exhibited greater uptake of bufotenin by human cells without attendant toxicity.

Literature-recommended qualitative (fluorescence microscopy) and quantitative (flow cytometry) confirmation of the formulated active plant ingredient (API) show augmented cellular uptake in comparison with the unformulated bufotenin solution. These results confirm that formulations A and B should be excellent delivery vehicle compositions for tryptamine-scaffold psychedelic agents.

The SNEDDS were prepared using an ethanol-assisted emulsification approach described in Banerjee et al., “Rational design, synthesis, and characterization of a solid A9-THC nano-formulation suitable for microdosing applications,” Cannabis Cannabinoid Res. 2023, 8(5), 1-13, the disclosure of which is hereby incorporated by reference. In this process, all ingredients (including the bufotenine as a representative tryptamine-scaffold psychedelic agent) were added to a round bottom flask and dissolved in ethanol by magnetic stirring at 750 rpm for 30 m. The ethanol was then removed through rotary evaporation (P=75 mbar; 2.5 h), producing a pre-emulsion product having the consistency of a viscous, syrupy liquid that solidifies in the refrigerator. Finally, HPLC-grade water was added to the flask of pre-emulsion product at 37° C. under gentle stirring to make an emulsion with the desired bufotenin concentration. The emulsions were stored in transparent vials at ˜4° C. The Nile Red/API co-loaded emulsions were made in the exact same way, with Nile Red solution in ethanol being added to the lipid phase.

In the first test, we looked into how much lipid can be loaded into the delivery composition of formula A at a constant Kolliphor (hydrophobic surfactant) and tween-80 (hydrophilic surfactant) amounts of 30 and 10 wt %, respectively without colloidal instability (measured by d_{z} exceeding 100 nm). In the lipid phase, API:MCT oil ratio (wt) was kept constant at 1:1. We found that a 20 wt % lipid payload (1:1 API:MCT oil) appeared optimal and was used as the loading level of choice for the next round of tests.

The second test varied the Kolliphor HS15 amount the optimal lipid loading determined in the earlier step. We determined that 20 wt % Kolliphor HS15 was our optimal concentration of this primary emulsifier.

The third test varied the Tween 80 amount to determine how much was needed to keep the droplet sizes below 100 nm. The API:MCT oil weight ratio was kept at constant 1:1. We found that 5 wt % was the optimal Tween-80 concentration to maintain the desired droplet sizes.

In order to determine lipid particle size distributions in the nano-emulsions, dynamic light scattering (DLS), also known as photon correlation spectroscopy, was applied. This technique generated Z-average diameters of the dispersed phase droplets (d_{z}), as well as the polydispersity index (PDI). Diluted samples (50- to 100-fold dilutions) were used to avoid multiple scattering. The measurements were conducted with the Zetasizer (Nano Z S, Malvern Instruments Ltd., UK). The Z-average diameters of the dispersed phase droplets was calculated from the autocorrelation function of the intensity of light scattered from the particles. Phase-separated nano-emulsions were re-mixed by shaking prior to dilution for DLS measurements.

### Effect of Adding α-Tocopherol

Given the instability of bufotenin as an API, we included the lipidic anti-oxidant additive, α-tocopherol, within formulation A to make formulation B. We found that the addition of α-tocopherol appeared to improve the colloidal properties of the resulting nano-emulsion upon dilution, with d_{z} changing from 90 nm (formulation A) to 35 nm (formulation B). The PDI of formulation B beneficially narrowed from 0.22 to 0.19 with an increased optical transparency.

As Table 1 shows, both formulations A and B form emulsions with average droplet sizes under 100 nm upon dissolution in water, a narrow range of nanoparticle sizes, and good loading levels. We expect that this basic formulation will produce nano-emulsions having an average droplet size within the range from about 10 nm to less than 100 nm, preferably within the range of about 20-95 nm, and even more preferably within the range of about 30-95 nm.

TABLE 1

| | Average Droplet | | Bufotenin |
|---|---|---|---|
| Formulation | Size (nm) | PDI | Loading (mg/g) |
| A | 90.1 ± 0.9 | 0.22 | 15.4 |
| B | 35.5 ± 1.2 | 0.19 | 25.6 |

### Stress Tests

The nano-emulsions of the present invention that were produced upon dissolution of the SNEDDS in water were stress-tested to examine the effect of storage times, heat, freeze/thaw cycles, and chemical additives on emulsion stability and API potency. The results show that the present nano-emulsion composition offered enhanced protection to bufotenin under conditions of simulated oxidative stress in comparison with unformulated bufotenin dissolved in ethanol. None of the stressors examined could induce phase separation in the emulsions created by dissolving the SNEDDS in water.

Cellular uptake studies showed quantitative penetration of glioma cells by the formulation. Cytotoxicity studies for these systems showed dilution dependent cytotoxicity to different cell lines, tapering to negligible toxicity values at therapeutic API doses.

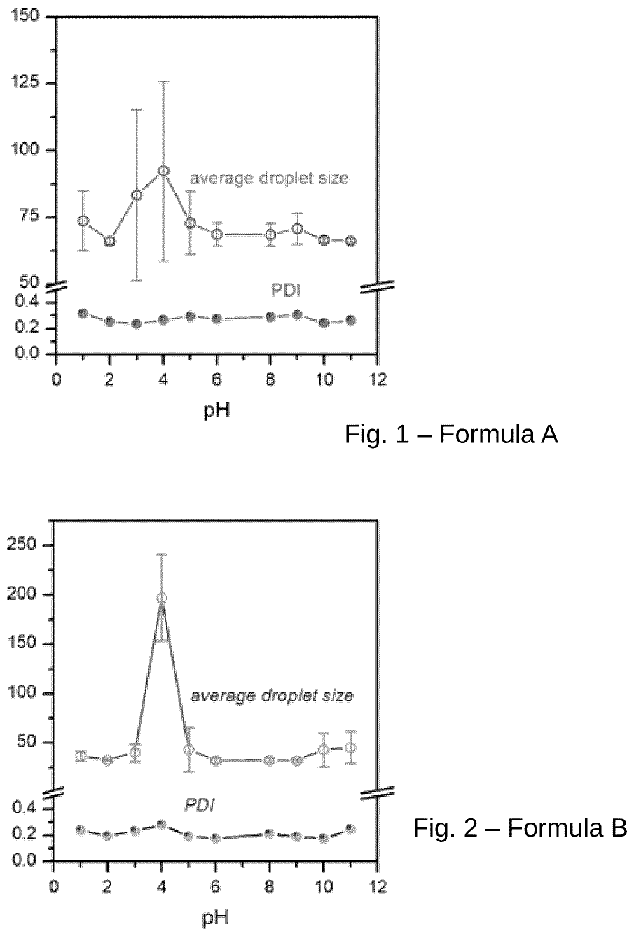

We also confirmed that bufotenin in an ethanolic solution is unstable under a variety of conditions that mimic commercial production conditions. This dictates that bufotenin must be specially formulated to protect the bufotenin from exposure and environmental stress. Testing of the nano-emulsion formulations A and B containing bufotenin proved that the emulsions are extremely stable even in the presence of stressors. Formulation A and B are colloidally stable at high and low pH values with only some droplet aggregation noticed between about pH 3 and 5. See Tables 1 and 2 as well as FIGS. 1 and 2.

TABLE 1
Formula A

| pH | dz (nm) | Error (nm) | PDI |
|---|---|---|---|
| 1 | 73.59 | 11.15 | 0.32 |
| 2 | 65.99 | 1.548 | 0.25 |
| 3 | 83.23 | 32.06 | 0.23 |
| 4 | 92.33 | 33.51 | 0.26 |
| 5 | 72.78 | 11.79 | 0.29 |
| 6 | 68.48 | 4.29 | 0.27 |
| 8 | 68.39 | 4.27 | 0.29 |
| 9 | 70.69 | 5.83 | 0.31 |
| 10 | 66.45 | 1.04 | 0.24 |
| 11 | 66.08 | 0.71 | 0.26 |

TABLE 2
Formula B

| pH | dz (nm) | Error (nm) | PDI |
|---|---|---|---|
| 1 | 36.60 | 5.18 | 0.24 |
| 2 | 32.78 | 1.13 | 0.20 |
| 3 | 40.01 | 8.97 | 0.23 |
| 4 | 197.1 | 43.70 | 0.28 |
| 5 | 43.35 | 22.37 | 0.19 |
| 6 | 32.16 | 2.13 | 0.17 |
| 8 | 32.38 | 1.94 | 0.21 |
| 9 | 32.00 | 1.57 | 0.19 |
| 10 | 43.17 | 16.92 | 0.17 |
| 11 | 45.35 | 16.21 | 0.25 |

Pro-oxidation stress tests: Bufotenin dissolved in ethanol (approx. 10-12 mg/g) showed between 50 and 60% API loss when exposed to various oxidation-promoting stressors. Upon exposing Formulas A and B to the same conditions, it was observed that the formulations offered moderate protection against bufotenin degradation in two of the three pro-oxidation environments studied. Heating at 37° C. for 8 hours led to ˜20% API loss in Formula A and ˜40% API loss in Formula B, but the unformulated bufotenin showed about 50% API degradation. Similarly, when exposed to the oxidation catalyst Cu(NO₃)₂(0.05 M) at 37° C. for 8 h, only Formula B showed less than 50% bufotenin loss. In the presence of peroxides, however, neither formula could prevent bufotenin degradation.

Biological studies on bufotenin: Untreated cells were run first to set the negative control as indicated by the absence of fluorescence. The cells treated with unformulated bufotenine and Nile Red were then examined. They appeared almost identical to the negative control, except for a slight shift to the positive fluorescence. We conclude that the unformulated API and dye in buffer are not taken up by the glioma cells. The cells treated with both of Formulas A and B showed about 100% cellular uptake. The fluorescent peak completely shifted to the zone of positive fluorescence, with no part of the curve even laying close to the negative/absent fluorescence region. Formulations A and B were both taken up by cells whereas the uptake of the control (Nile Red and unformulated bufotenin suspended in the buffer) was negligible. These results further demonstrate that the nano-emulsions of the present invention can increase the permeability and liquidity of the membrane, promote the transmembrane transfer of the API, and increase the amount of cell intake of the API.

Cytotoxicity evaluation: our tests show a dilution-dependent cytotoxicity of Formulas A and B. The unformulated bufotenin is not taken up by the cells and thus has no effect on their viability.

### Experimental Details—Materials

All materials were purchased from Sigma Aldrich unless otherwise stated, and used as received. bufotenin was synthesized in-house by the Trant organic synthesis lab and used as received. MCT oil and tween 80 was purchased from Charles Tennent company Ltd., Canada. HPLC-grade water (EMD Millipore) was used in all experiments. CaCl₂ and sucrose were purchased from Sigma Aldrich. Tocopherol and potassium sorbate were purchased from Caldic. Measurement of pH was done on a freshly calibrated daily pH meter (Milwaukee MW102 PRO+).

Four different cell lines were used for the evaluation of the tested formulations and unformulated bufotenin.
L2:Rat lung cells (Normal) https://www.atcc.org/products/ccl-149
SW982: The human synovial sarcoma cells https://www.atcc.org/products/htb-93
HCT116: The human colon cancer cells https://www.atcc.org/products/ccl-247
231: The human breast cancer cells https://www.atcc.org/products/crm-htb-26

### Preparation of the Bufotenin Nano-Emulsions

The nano-emulsions of Formulas A and B were prepared using ethanol-assisted emulsification. All the ingredients were added to a round bottom flask and dissolved in ethanol by magnetic stirring at 750 rpm for 30 m. The ethanol was then removed through rotary evaporation (P=75 mbar; 2.5 h) to produce a viscous, syrupy system that solidifies in the refrigerator. These are the SNEDDS. Finally, HPLC-grade water was added to the flask at 37° C. under gentle stirring to make an emulsion with the desired bufotenin concentration. The emulsions were stored in transparent vials at ˜4° C. The Nile Red/API co-loaded emulsions were made in the exact same way, with Nile Red solution in ethanol being added to the lipid phase.

Long-term storage: To examine the effect of long-term storage on the colloidal and chemical stability of the nano-emulsion, we stored them in tightly capped amber glass vials in a refrigerator at 4° C. Aliquots were periodically removed from the vials and diluted prior to DLS measurements. Measurements were performed immediately after high pressure homogenization, and after that, once every seven days for up to six weeks.

Flash heating: 1 g of the optimized nano-emulsion was placed in a preheated water bath and the internal temperature of the nano-emulsion was maintained at 80° C. for 1 minute. This protocol is a more extreme version of the high-temperature short-time (HTST) pasteurization process (typically, 71.5° C. for 15 s) that fruit juices and milk beverages are subjected to in the industry. The nano-emulsion was then allowed to cool to room temperature, and a part of it was diluted for DLS study. The rest was retained for HPLC analysis.

Freeze-thaw cycle: 1 g of the optimized nano-emulsion was placed in a freezer at a temperature of −20° C. for 1 hour. The nano-emulsion was then removed from the freezer and allowed to revert to room temperature. A part of the thawed nano-emulsion was diluted for DLS study, and the rest was retained for HPLC analysis.

Additives: In a representative experiment, varying masses of CaCl₂·2H₂O, sucrose, and potassium sorbate were added individually to 1 g portions of the nano-emulsions at certain pre-determined concentrations. To ensure complete dissolution of the additive, the aliquots were vortexed for 1 min each. After an incubation period of 12-18 h, the aliquots were vortexed again for 1 min prior to dilution with DLS studies. It is to be noted that HPLC analysis studies were not carried out in the context of salt, sugar and preservative addition to the nano-emulsions, given that they are not expected to degrade bufotenin in any meaningful way. Any drop in bufotenin for these experiments may be attributed to colloidal events leading to instability rather than to chemical transformations.

Extraction procedure: 30-50 mg of emulsion samples were weighed individually into 15 mL falcon tubes and 5 mL methanol (HPLC grade) were added to the samples. Samples were vortexed for 10 s and placed in an ultrasonic bath for 15 min at room temperature. After the sonication, sample was spun at 400×g for 5 min in a centrifuge (Avantor). Aliquots of resultant clear supernatant were used for HPLC analysis.

Sample acquisition and data analysis: Chromatographic analysis of supernatants from sample extraction was performed using an Agilent 1260 Liquid Chromatography system fitted with a photo diode array detector and using an Agilent Zorbax Eclipse XDB-C18 column (4.6×150 mm, 5 μm). For data acquisition, the following gradient run was applied using a mobile phase combination of water/0.05% trifluoroacetic acid (A) and acetonitrile (B): 90% CH₃CN for 0-4 m, 50% CH₃CN for 5-9 m, and 90% CH₃CN for 10-13 m. Flow rate was set at 0.5 mL·min⁻¹, and the analyte detected at 280 nm. Mobile phase solvents were of HPLC grade and filtered with 0.20 μm filters prior to analysis. Standard dilutions of bufotenin from 0.64 mL·min⁻¹ to 0.0064 mL-min⁻¹ were run alongside sample extracts and data generated from acquisition used to plot a calibration curve to calculate concentration of bufotenin in samples.

Cellular uptake studies: The human glioma U251 GBM cells were cultured at 5×10⁴ cells per well in 12 plates using EMEM media supplemented with 10% FBS, 0.5% PenStrep and 1% w/non-essential amino acids (NEAA); then, washed with 0.05% trypsin. The cells were cells were incubated overnight at 37° C. in a humidified 5% CO₂ atmosphere. The cells were treated with NEs loaded with bufotenine and Nile Red dye diluted to 0.1 mg/ml in PBS. Untreated cells were used as a negative control a long with a solution of unformulated API and Nile Red. This was done to serve as a sort of a positive control against the negative control of the untreated cells. After 24 h exposure, cells were washed twice with PBS and then collected in PBS-EDTA media (2 mM). The flow cytometry was performed using a BD C6 Flow Cytometry apparatus at a wavelength of 488 nm laser. For each experiment 10,000 cells were gated per sample and each sample was performed in triplicate. These cells prepared as such were used for fluorescence analysis. Fluorescence images were taken using a Leica CRT 6500 at the desired magnification using Texas Red Set (Leica TX2 ET Set) filter and exciting the samples using a continuous wave Ar+ laser at 488 nm.

Cytotoxicity assay: The effect of the NEs on cell viability was measured by MTT assay following the method by Mosmann and colleagues. Briefly, the cells were seeded in a 96 well micro titer plate (100 μL per well) with replications. After 24 h, cells were treated with 50 μL of the analyte at four different dilutions (10,100,1000, and 10000-fold), added directly to wells with culture media. The starting concentration for Kolliphor-Bf-SNEDDS, Kolliphor-Bfₐ₋ₜ-SNEDDSand unformulated bufoteninin DMSO were 1 mg·g⁻¹. After incubation for 48 h, the media was discarded. 100 μL of 0.5 mg·mL⁻¹ MTT stock solution was added to each well and incubated for 4 h at 37° C. The obtained formazan crystals were solubilized with DMSO, and the absorbance was measured at 570 nm using a microplate reader (SpectraMax M5e, Molecular Devices, USA). Cell viability (%) was calculated as a ratio of absorbance (A570) in treated cells to absorbance in control cells (intact cells without treatment) (A570).

Statistical analysis: All DLS measurements were carried out in triplicate on at least two samples. Mean and standard deviations were calculated using Microsoft's Excel spreadsheet package (2016). For the stress tests, two replicate studies were performed, and either the average of the measured values used, or both values plotted on the relevant graph.

1. An oil-in-water nano-emulsion comprising: (a) a therapeutic amount of one or more tryptamine-scaffold psychedelics, (b) an organic vehicle comprising a trigyceride, (c) a first hydrophilic surfactant, (d) a second surfactant comprising polyethylene glycol 660 12-hydoxystearate, and, optionally, (e) an anti-oxidant comprising Vitamin E.

2. A nano-emulsion as in claim 1 wherein said composition further comprises a lower alkyl alcohol.

3. A nano-emulsion as in claim 2 wherein said lower chain alcohol comprises ethanol.

4. A nano-emulsion as in claim 1 wherein said sorbitan monooleate comprises polyoxyethylene (80) sorbitan monooleate.

5. A nano-emulsion according to claim 1 wherein nonionic surfactant comprises about 70% of polyglycol mono- and di-esters (lipophilic) and about 30% of free polyethylene glycol.

6. A nano-emulsion according to claim 1 further comprising a lipidic antioxidant.

7. A nano-emulsion according to claim 5 wherein said antioxidant comprises α-tocopherol.

8. A nano-emulsion according to claim 1 wherein said tryptamine-scaffold psychedelic comprises psilocybin, mescaline, psilocin, ibogaine, or bufotenin.

9. A nano-emulsion according to claim 8 wherein said tryptamine-scaffold psychedelic comprises psilocybin, psilocin, or bufotenin.

10. A nano-emulsion according to claim 9 wherein said tryptamine-scaffold psychedelic comprises bufotenin.

11. A nano-emulsion according to claim 1 having an average droplet size in said emulsion that is within the range from about 10 nm to less than 100 nm.

12. A nano-emulsion according to claim 11 wherein the average droplet size is within the range from about 20-95 nm.

13. A nano-emulsion according to claim 12 wherein the average droplet size is within the range from about 30-95 nm.

14. A nano-emulsion according to claim 1 in liquid form and having a concentration of tryptamine-scaffold psychedelic agent an amount within the range from about 0.1-1000 mg/ml.

15. A nano-emulsion according to claim 14 comprising a concentration of tryptamine-scaffold psychedelic agent in an amount within the range of 1-100 mg/ml.

16. A process for the manufacture of the SNEDDS of the present invention by a process that comprises:
(a) combining (i) a therapeutic amount of one or more tryptamine-scaffold psychedelics, (ii) a triglyceride oil, (iii) a hydrophilic surfactant, (iv) a nonionic surfactant, and, optionally, (v) an anti-oxidant comprising Vitamin E in a container,
(b) adding a lower chain alcohol to said container while mixing the ingredients therein to form a pre-emulsion product,
(c) optionally cooling said pre-emulsion product, and
(d) diluting the pre-emulsion product with water to form a self-nanoemulsifying drug delivery system.

17. A process for delivering a therapeutic amount of one or more tryptamine-scaffold psychedelics to a human or mammal patient by a process that comprises: spraying into a nasal cavity of said patient a metered amount of a composition comprising (a) a therapeutic amount of one or more tryptamine-scaffold psychedelics, (b) a trigyceride oil, (c) a first hydrophilic surfactant, (d) a second surfactant comprising polyethylene glycol 660 12-hydoxystearate, and, optionally, (e) an anti-oxidant comprising Vitamin E.

18. A non-aqueous, pre-emulsion composition comprising a therapeutic amount of a tryptamine-scaffold psychedelic drug and one or more surfactants, wherein the composition is capable of self-emulsifying upon mixture with an aqueous solution.

19. A non-aqueous, pre-emulsion composition according to claim 18 further comprising a triglyceride oil, a hydrophilic surfactant, and a second surfactant comprising polyethylene glycol 660 12-hydoxystearate.

20. A non-aqueous, pre-emulsion composition according to claim 18 further comprising an antioxidant comprising Vitamin E.
